(19) United States
(12) Patent Application Publication
(10) Pub. No.: US 2021/0355451 A1
(43) Pub. Date: Nov. 18, 2021

(54) VIABLE BIOENGINEERED SKIN CONSTRUCTS

(71) Applicant: Stratatech Corporation, Madison, WI (US)

(72) Inventors: B. Lynn Allen-Hoffmann, Madison, WI (US); Allen R. Comer, Madison, WI (US); Kenneth R. Gratz, Madison, WI (US); Barry M. Steiglitz, Madison, WI (US)

(21) Appl. No.: 17/319,760

(22) Filed: May 13, 2021

Related U.S. Application Data
(60) Provisional application No. 63/024,258, filed on May 13, 2020.

(51) Int. Cl. C12N 5/071; A61K 35/12; A61K 38/39

(52) U.S. Cl. CPC C12N 5/0698; A61K 35/12; C12N 2502/1323; C12N 2500/90; C12N 2502/094; A61K 38/39

(57) ABSTRACT

The present disclosure is directed to viable bioengineered skin constructs.

### CROSS REFERENCE TO RELATED APPLICATIONS

The present application claims priority to U.S. Provisional Appl. No. 63/024,258, filed May 13, 2020, herein incorporated by reference in its entirety.

### FIELD OF THE DISCLOSURE

The present disclosure is directed to viable bioengineered skin constructs.

### BACKGROUND OF THE DISCLOSURE

Potential solutions to wound healing often include skin substitutes. These substitute products may be used to help close and protect wounds. Often, however, the substitute products leave much to be desired, particularly when it comes to the physical characteristics of the substitute, such as tensile strength. There is a need in the art, therefore, for substitute products with physical characteristics that promote durability of the substitute and provide significant benefit to the wound.

### SUMMARY OF THE DISCLOSURE

One aspect of the present disclosure encompasses a viable, bioengineered skin construct comprising a fully stratified epithelial layer having a top surface and a bottom surface, wherein the fully stratified epithelial layer comprises human keratinocytes, and a dermal equivalent layer having a top surface and a bottom surface, wherein the dermal equivalent layer comprises human dermal fibroblasts within a matrix. The matrix comprises human collagen and optionally murine type I collagen. The bottom surface of the epithelial layer is adhered to the top surface of the dermal equivalent layer over at least 98% of one of the layers. A dog bone-shaped sample of the skin construct fails at a load of about 0.5 N to about 1.0 N (“the failure load”) and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm; wherein the dog bone-shaped sample has a 4 mm gauge width and a 25 mm gage length, as measured by a thickness gauge.

Another aspect of the present disclosure encompasses a viable, bioengineered skin construct comprising a fully stratified epithelial layer having a top surface and a bottom surface, wherein the fully stratified epithelial layer comprises human keratinocytes, and a dermal equivalent layer having a top surface and a bottom surface, wherein the bottom surface of the epithelial layer is adhered to the top surface of the dermal equivalent layer over at least 98% of one of the layers. The dermal equivalent layer comprises human dermal fibroblasts within a matrix, the matrix comprising human type I collagen, human type III collagen, human type IV collagen, and human type VI collagen and optionally murine type I collagen. The skin construct has a total collagen content of about 0.25 mg per cm² of surface area to about 0.45 mg per cm² of surface area and at least 0.05 mg per cm² of surface area of human type I collagen. A dog bone-shaped sample of the skin construct fails at a load of about 0.5 N to about 1.0 N and a displacement of about 30 mm to about 45 mm when pulled to failure in uniaxial tension at a constant strain rate of 100% per minute with continuous hydration using Dulbecco's phosphate-buffered saline. The fully stratified epithelial layer fails at the same point in the load-displacement curve as the skin construct and the dermal equivalent layer fails at a displacement of about 12-18 mm or about 14-16 mm. The dog bone-shaped sample has a 4 mm gauge width and a 25 mm gage length, as measured by a thickness gauge

Yet another aspect of the present disclosure encompasses a viable, bioengineered skin construct comprising a fully stratified epithelial layer having a top surface and a bottom surface, wherein the fully stratified epithelial layer comprises NIKS cells, and a dermal equivalent layer having a top surface and a bottom surface, wherein the bottom surface of the epithelial layer is adhered to the top surface of the dermal equivalent layer over at least 98% of one of the layers. The dermal equivalent layer comprises normal human dermal fibroblasts within a matrix, the matrix comprising human type I collagen, human type III collagen, human type IV collagen, and human type VI collagen and optionally murine type I collagen. The skin construct has a total collagen content of about 0.25 mg per cm² of surface area to about 0.45 mg per cm² of surface area and at least 0.05 mg per cm² of surface area of human type I collagen. A dog bone-shaped sample of the skin construct fails at a load of about 0.5 N to about 1.0 N and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm. The dog bone-shaped sample has a 4 mm gauge width and a 25 mm gage length, as measured by a thickness gauge.

Still another aspect of the present disclosure encompasses a viable, bioengineered skin construct comprising a fully stratified epithelial layer having a top surface and a bottom surface, wherein the fully stratified epithelial layer comprises human keratinocytes and a dermal equivalent layer having a top surface and a bottom surface, wherein the bottom surface of the epithelial layer is adhered to the top surface of the dermal equivalent layer over at least 98% of one of the layers.

The dermal equivalent layer comprises human dermal fibroblasts within a matrix, the matrix comprising human type I collagen, human type III collagen, human type IV collagen, and human type VI collagen and optionally murine type I collagen. The skin construct has a total collagen content of about 0.25 mg per cm² of surface area to about 0.45 mg per cm² of surface area and at least 0.05 mg per cm² of surface area of human type I collagen.

Some aspects of the present disclosure encompass a viable, bioengineered skin construct comprising a fully stratified epithelial layer having a top surface and a bottom surface, wherein the fully stratified epithelial layer comprises NIKS cells, and a dermal equivalent layer having a top surface and a bottom surface, wherein the bottom surface of the epithelial layer is adhered to the top surface of the dermal equivalent layer over at least 98% of one of the layers. The dermal equivalent layer comprises normal human dermal fibroblasts within a matrix, the matrix comprising human type I collagen, human type III collagen, human type IV collagen, and human type VI collagen and optionally murine type I collagen. The skin construct has a total collagen content of about 0.25 mg per cm² of surface area to about 0.45 mg per cm² of surface area and at least 0.05 mg per cm² of surface area of human type I collagen.

Other aspects of the present disclosure encompass a viable, bioengineered skin construct comprising a fully stratified epithelial layer having a top surface and a bottom surface, wherein the fully stratified epithelial layer comprises human keratinocytes, and a dermal equivalent layer having a top surface and a bottom surface, wherein the dermal equivalent layer comprises human dermal fibroblasts within a matrix. The matrix comprises human collagen and optionally murine type I collagen. The skin construct has a total collagen content of about 0.25 mg per cm² of surface area to about 0.45 mg per cm² of surface area and at least 0.05 mg per cm² of surface area of human type I collagen. A dog bone-shaped sample of the skin construct fails at a load of about 0.5 N to about 1.0 N (“the failure load”) and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm; wherein the dog bone-shaped sample has a 4 mm gauge width and a 25 mm gage length, as measured by a thickness gauge.

Each of the above aspects, along with additional aspects and iterations of the present disclosure are detailed herein.

### BRIEF DESCRIPTION OF THE FIGURES

FIG. 1 is a graph illustrating a representative load-displacement curve of StrataGraft® during tensile testing.

FIG. 2 is a diagram illustrating the sample dimensions for tensile testing.

### DETAILED DESCRIPTION

A viable, bioengineered skin construct of the present disclosure encompasses a fully stratified epithelial layer and a dermal equivalent layer. A skin construct of the present disclosure may be used as an organotypic human skin equivalent. Accordingly, the skin construct is a bioengineered (non-natural) bilayer tissue designed to mimic natural human skin with both an inner dermis-like layer and an outer epidermis-like layer. For example, production of the skin construct by organotypic culture produces a well-developed epidermal layer of fully-stratified human keratinocytes that exhibits barrier function comparable to that of intact human skin.

The viable cells of the skin substitute (e.g., fibroblasts, NIKS cells, etc.) are metabolically active and secrete a spectrum of growth factors, chemotactic factors, cytokines, inflammatory mediators, enzymes, and host defense peptides that, after the skin construct is applied to a wound, may condition the wound bed, promote tissue regeneration and repair, and reduce infection. In an exemplary embodiment, the skin construct is StrataGraft®™. In another exemplary embodiment, the skin construct is ExpressGraft™.

In some embodiments, a skin construct has a thickness of about 100 μm to about 250 μm, or about 120 μm to about 200 μm, as measured by histology. For instance, a skin construct may have a thickness of about 100, 110, 120, 130, 140, 150, 160, 170, 180, 190, 200, 210, 220, 230, 240, or 250 μm, as measured by histology.

Generally speaking, a skin construct of the current disclosure may comprise any desired dimensions and surface area, limited only by the culture plates utilized. In particular embodiments, a skin construct of the present disclosure has a surface area of about 20 cm² to about 250 cm². In some embodiments, a skin construct of the present disclosure has a surface area of about 20 cm² to about 80 cm², about 80 cm² to about 140 cm², about 140 cm² to about 200 cm², or about 200 cm² to about 250 cm². In certain embodiments, a skin construct of the present disclosure has a surface area of about 90 cm² to about 110 cm². In one embodiment, a skin construct of the present disclosure has a surface area of about 100 cm².

A skin construct of the present disclosure may optionally be meshed. In some embodiments, a skin construct has a mesh ratio of about 1:1 or more (e.g., about 1.5:1, about 2:1, about 2.5:1, about 3:1, etc.). In some embodiments, a skin construct is not meshed.

Each of the layers of the skin construct is detailed below, along with other defining characteristics of the present skin constructs.

### (a) Fully Stratified Epithelial Layer

A skin construct of the present disclosure comprises a fully stratified epithelial layer that is epidermis-like. The fully stratified epithelial layer has a top surface and a bottom surface, and comprises human keratinocytes.

In some embodiments, the fully stratified epithelial layer comprises NIKS cells. NIKS® cells were deposited with the ATCC (CRL-12191) and are described in further detail in U.S. Pat. Nos. 5,989,837 and 6,964,869, the disclosures of which are incorporated herein by reference.

A fully stratified epithelial layer may encompass NIKS® cells engineered to express a variety of exogenous nucleic acids. Expressly contemplated are NIKS® cells engineered to express an exogenous gene encoding a VEGF protein (e.g., VEGF-A, etc.), an exogenous gene encoding a hypoxia-inducible factor (e.g., HIF-1A, etc.), an exogenous gene encoding an angiopoietin (e.g., ANGPT1, etc.), an exogenous gene encoding a cathelicidin peptide or a cleavage product thereof (e.g., hCAP-18, etc.), an exogenous gene encoding a beta-defensin (e.g., hBD-3, etc.), an exogenous gene encoding a keratinocyte growth factor (e.g., KGF-2, etc.), an exogenous gene encoding a tissue inhibitor of metalloproteinases (e.g., TIMP-1, etc.), an exogenous IL-12 gene, as well as exogenous nucleic acid sequences encoding other antimicrobials, growth factors, transcription factors, interleukins and extracellular matrix proteins. As non-limiting examples, see for instance, U.S. Pat. Nos. 7,498,167, 7,915,042, 7,807,148, 7,988,959, 8,808,685, 7,674,291, 8,092,531, 8,790,636, 9,526,748, 9,216,202, and 9,163,076, and US 20190030130, the disclosures of which are incorporated herein by reference. Skin constructs comprising NIKS cells engineered to express an exogenous nucleic acid encoding a desired protein produce a greater amount of that protein (e.g., at least 10%, at least 20%, at least 30%, etc. more) than a skin construct comprising NIKS cells that do not contain the exogenous nucleic acid.

In some embodiments, the fully stratified epithelial layer has a thickness of about 75 μm to about 120 μm, as measured by histology. For example, the fully stratified epithelial layer may have a thickness of about 75, 80, 85, 90, 95, 100, 105, 110, 115, or 120 μm, as measured by histology.

### (b) a Dermal Equivalent Layer

A skin construct of the present disclosure encompasses a dermal equivalent layer that is dermis-like. The dermal equivalent layer has a top surface and a bottom surface, and comprises human dermal fibroblasts within a matrix.

In some embodiments, the dermal equivalent layer has a thickness of about 20 μm to about 80 μm, as measured by histology. For example, the dermal equivalent layer may have a thickness of about 20, 25, 30, 35, 40, 45, 50, 55, 60, 65, 70, 75, or 80 μm, as measured by histology.

i. Human Dermal Fibroblasts

A skin construct of the present disclosure encompasses a dermal equivalent layer that comprises human dermal fibroblasts. In exemplary embodiments, the human dermal fibroblasts are primary normal human dermal fibroblasts. In some embodiments, the human dermal fibroblasts are immortalized.

ii. Matrix

A skin construct of the present disclosure encompasses a dermal equivalent layer that comprises a matrix. The matrix of the dermal equivalent layer comprises human collagen and optionally, murine type I collagen. In some embodiments, the matrix of the dermal equivalent layer comprises human type I collagen, human type III collagen, human type IV collagen, human type VI collagen, and optionally, murine type I collagen.

The collagen present in the dermal equivalent may include type I murine collagen. Alternatively, the only collagen present in the dermal equivalent may be produced by cells of the skin substitute (e.g., human dermal fibroblasts). The matrix may further comprise additional biomolecules produced by the cells contained therein. In an exemplary embodiment, the dermal layer is composed of normal human dermal fibroblasts embedded within a matrix produced and organized by the fibroblasts (e.g. an extracellular matrix). In some iterations of this embodiment, there is no non-human collagen in the dermal equivalent layer. In other iterations of this embodiment, there is up to about 85% non-human collagen in the dermal equivalent layer. In particular iterations, the non-human collagen is murine. In another exemplary embodiment, the dermal equivalent layer is composed of normal human dermal fibroblasts embedded in a gelled-collagen matrix that contains purified murine type I collagen. For the avoidance of doubt, in this embodiment, although the murine type I collagen is gelled to give the dermal layer its primary structure, the normal human dermal fibroblasts embedded therein may produce and contribute collagen (and other biomolecules) to the matrix.

iii. Cell Combinations

A skin construct of any of the above embodiments may comprise keratinocytes of the epithelial layer from a single human donor or dermal fibroblasts of the dermal equivalent layer from a single human donor. Alternatively, a skin construct of any of the above embodiments may comprise keratinocytes of the epithelial layer from a single human donor and dermal fibroblasts of the dermal equivalent layer from a single human donor. Optionally, the donor of the dermal fibroblasts may be different from the human donor of the keratinocytes.

In some embodiments, a skin construct of the present disclosure may comprise keratinocytes that are NIKS cells or dermal fibroblasts that are normal human dermal fibroblasts. In other embodiments, a skin construct of the present disclosure may comprise keratinocytes that are NIKS cells and dermal fibroblasts that are normal human dermal fibroblasts.

### (c) Adherence

A skin construct of the present disclosure comprises two layers as detailed above—an epithelial layer and a dermal equivalent layer. In all instances, the bottom surface of the epithelial layer is adhered to the top surface of the dermal equivalent layer over at least 85% of one of the layers. In certain embodiments, the bottom surface of the epithelial layer is adhered to the top surface of the dermal equivalent layer over at least 85%, 86%, 87%, 88%, 89%, 90%, 91%, 92%, 93%, 94%, 95%, 96%, 97%, 98%, 99%, or greater than 99% of one of the layers. In some embodiments, the bottom surface of the epithelial layer is adhered to the top surface of the dermal equivalent layer over at least 95%, 96%, 97%, 98%, 99%, or greater than 99% of one of the layers. In particular embodiments, the bottom surface of the epithelial layer is adhered to the top surface of the dermal equivalent layer over at least 98%, 99%, or greater than 99% of one of the layers.

As used herein, “adhered” refers to the natural interactions between the layers that occurs during manufacturing, and does not refer to any sort of artificial glue or adhesive.

### (d) Collagen

A skin construct of the present disclosure has a total collagen content of about 0.20 mg per cm² of skin construct surface area to about 0.50 mg per cm² of skin construct surface area as measured using the protocol detailed in the Examples below. In some embodiments, a skin construct may have a total collagen content of about 0.25 mg per cm² of skin construct surface area to about 0.45 mg per cm² of skin construct surface area. For instance, a skin construct may have a total collagen content of about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, or 0.45 mg per cm² of skin construct surface area. In particular embodiments, a skin construct may have a total collagen content of about 0.25 to about 0.30, about 0.30 to about 0.35, about 0.35 to about 0.40, or about 0.40 to about 0.45 cm² of skin construct surface area.

In particular embodiments, a skin construct of the present disclosure comprises human Type 1 collagen. For instance, a skin construct of the present disclosure may have at least 0.05 mg per cm² of surface area of human type I collagen, as measured using the protocol detailed in the Examples below. In some embodiments, a skin construct has at least 5 mg of human type I collagen per 100 cm². For example, a skin construct may have at least 5.5 mg of human type I collagen or at least at least 5.8 mg of human type I collagen per 100 cm².

In alternative embodiments, a skin construct of the present invention may have at least 0.055 mg of human type I collagen per cm² of surface area or at least 0.058 mg of human type I collagen per cm² of surface area.

In each of the above embodiments encompassing a skin construct of the present disclosure comprising human type 1 collagen, about 95% or more of the human type I collagen is produced by cells of the skin construct. For instance, about 95, 96, 97, 98, 99, or 100% of the human type I collagen may be produced by cells of the skin construct. In particular embodiments, about 100% of the human type I collagen is produced by cells of the skin construct.

A skin construct of the present disclosure may comprise human type I collagen and murine type I collagen, wherein the murine type I collagen is not more than 90% by weight of total collagen in the skin construct. For instance, in some embodiments, the murine type 1 collagen is about 60% to about 90% by weight of the total type collagen in the skin construct.

In preferred embodiments, a skin construct of the present disclosure has a total collagen content of about 0.25 mg per cm² of surface area to about 0.45 mg per cm² of surface area and at least 0.05 mg per cm² of surface area of human type I collagen.

In certain embodiments, a skin construct of the present disclosure may comprise up to about 88% murine collagen. For instance, a skin construct of the present disclosure may comprise up to about 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, or 50% murine collagen. In each of the embodiments described in this paragraph, the non-murine collagen is human collagen.

In particular embodiments, a skin construct of the present disclosure may comprise about 10 to about 25% human collagen by mass. In these embodiments, the human collagen is derived from normal human dermal fibroblasts. For instance, a skin construct of the present disclosure may comprise about 10, 11, 12, 13, 14, 15, 16, 17, 18, 19, 20, 21, 22, 23, 24 or 25% human collagen by mass. In certain embodiments, a skin construct of the present disclosure may comprise about 15 to about 20% human collagen by mass.

### (e) Failure Load

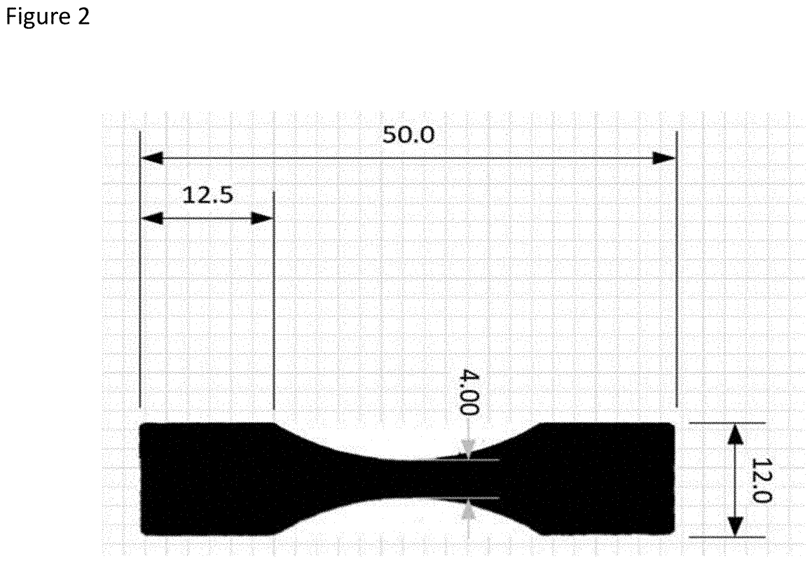

A skin construct of the present invention has a failure load of between about 0.5N to about 1.0N and a displacement of about 30 mm to about 45 mm. As used herein, “failure load” refers to the force required to pull a sample of the skin construct to failure in uniaxial tension at a constant strain rate of 100% per minute with continuous hydration using Dulbecco's phosphate-buffered saline, wherein the sample is shaped like a symmetrical dog bone with a gauge width of 4 mm and a gauge length of 25 mm. For example, see the illustration of an appropriate sample in FIG. 2.

In some embodiments, a skin construct of the present invention has a failure load of about 0.5N, 0.6N, 0.7N, 0.8N, 0.9N, or 1.0N and a displacement of about 30 mm to about 45 mm. In certain embodiments, a skin construct of the present invention has a failure load of between about 0.5N to about 1.0N and a displacement of about 30, 31, 32, 33, 34, 35, 36, 37, 38, 39, 40, 41, 42, 43, 44, or 45 mm.

A skin construct of the present disclosure fails in two phases: the dermal equivalent layer of the skin construct fails first, followed by the fully stratified epithelial layer. For instance, in some embodiments the dermal equivalent layer fails at a displacement of about 12-18 mm or about 14-16 mm. In certain embodiments, the dermal equivalent layer fails at a displacement of about 12, 13, 14, 15, 16, 17, or 18 mm. In some embodiments, the dermal equivalent layer fails at a displacement of about 14-16 mm and a load of about 0.1 N to about 0.5 N.

In certain embodiments, the load drops less than 10% of the failure load when the dermal equivalent layer fails. For instance, the load may drop less than 10%, 9%, 8%, 7%, 6%, 5%, 4%, 3%, 2% or 1% of the failure load when the dermal equivalent layer fails. In one embodiment, the load drops less than 1% of the failure load when the dermal equivalent layer fails.

### (f) Manufacturing

Suitable manufacturing processes for producing a skin construct have been previously described in the art. See, for instance, U.S. Pat. Nos. 7,498,167, 7,915,042, 7,807,148, 7,988,959, 8,808,685, 7,674,291, 8,092,531, 8,790,636, 9,526,748, 9,216,202, 9,163,076, 10,091,983, and US 20190030130, the disclosures of which are each incorporated by reference in their entirety.

A skin construct of the present disclosure may be cryopreserved. Methods of cryopreservation are known in the art. See, for instance, U.S. Pat. No. 10,091,983, herein incorporated by reference in its entirety.

After a skin construct of the present disclosure is thawed following cryopreservation, the skin construct may secrete a plurality of proteins selected from bFGF, GM-CSF, HGF, IL-1α, IL-6, IL-8, IL-10, MMP-1, MMP-3, MMP-9, PIGF, SDF-1α, TGF-β1, and VEGF-A.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Introduction

Studies were undertaken to examine expression of extracellular matrix (ECM) molecules within StrataGraft® tissue after finalizing manufacturing processes. While the nascent dermal equivalent (DE) has a defined composition at the formulation step, consisting of input murine type I collagen and normal human dermal fibroblasts (NHDF), the ECM composition of mature StrataGraft® was investigated to assess changes in DE composition during the organotypic stage of StrataGraft® skin tissue manufacture. The studies conducted demonstrate that the DE undergoes significant changes during the StrataGraft® manufacturing process, through elaboration of human ECM molecules from NIKS keratinocytes and NHDF.

The manufacturing process for StrataGraft® skin tissue encompasses three sequential cell and tissue culture processes. In Stage I of the manufacturing process, NIKS keratinocytes are expanded in monolayer cell culture. Concurrent with the NIKS keratinocyte culture in Stage I, NHDF are expanded in monolayer culture and combined with purified type I collagen and culture medium and allowed to gel to form the cellularized dermal equivalent (DE). In Stage II, NIKS keratinocytes are seeded onto the surface of the DE and cultured under submerged conditions for two days to promote complete epithelialization of the DE surface. The tissue is then lifted to the air-liquid interface in Stage III, where it is maintained for 18 days in a controlled, low humidity environment to promote tissue maturation. The skin equivalents are generally prepared as described in U.S. Pat. Nos. 7,674,291; 7,807,148; 7,915,042; 7,988,959; and 8,092,531; each of which is incorporated herein by reference in its entirety.

Based on results of ELISA studies, the total amount of human type I collagen synthesized in one 100 cm² StrataGraft® tissue is estimated to be 5.8 mg per tissue at the notional drug substance stage, just prior to cryopreservation. Quantification of hydroxyproline content indicates a total collagen content of 29.6 to 38.6 mg in each mature 100 cm² StrataGraft® tissue. This indicates that 15 to 20% of collagen by mass is human collagen derived from NHDF and the remaining 80 to 85% of collagen by mass is of murine origin. Based on an input mass of 51.5 mg murine collagen, it has been determined that approximately 37 to 54% of the input murine collagen may be eliminated during the StrataGraft® manufacturing process. Despite residual murine collagen in StrataGraft®, data presented here suggest that throughout the StrataGraft® manufacturing process, the nascent DE which is formulated using murine type I collagen embedded with NHDF, is transformed by the production and assembly of many of the major structural and functional ECM elements of human skin. Data herein show that while the exogenous murine collagen gel provides an initial physiological substrate for the input NHDF and NIKS cells, it does not contribute substantially to the final mechanical properties of the tissues. The modification and reorganization of the ECM parallels the changes in the overall DE structure, which starts as a loosely organized 2.3 mm thick (23 mL volume) hydrogel and transitions to a more compact <100 μm thick (1 mL volume) layer in the mature tissue.

Importantly, both NIKS keratinocytes and NHDF are believed to bind collagen via integrin receptors and discoidin domain receptors. This binding elicits biological responses within the keratinocytes and the fibroblasts during the development of the StrataGraft® tissue, resulting in unique tensile characteristics of the finished 100 cm² skin construct. The input murine type I collagen starting material provides a biologically relevant environment in which cellular maturation and paracrine signaling between the NIKS keratinocytes and NHDF are enabled. The collagen promotes initial cellular adhesion, protein secretion, proliferation, and differentiation of the input NIKS keratinocytes and NHDF. The organotypic culture environment promotes the formation and maintenance of a structurally organized ECM comprising other collagens, adhesion proteins, proteoglycans, and matrix-bound enzymes and growth factors. Type I collagen is also anticipated to serve as a functional regulator of cellular activities within the developing StrataGraft® tissue.

The studies summarized here employed the use of indirect immunofluorescence (IIF) on thin cross-sections of StrataGraft® skin tissue at various time points during the StrataGraft® skin tissue manufacture, and ELISA of conditioned media harvested from StrataGraft® skin tissue growth chambers during the manufacturing process in order to track synthesis and accumulation of human ECM during StrataGraft® manufacture. This analysis demonstrates that the initial collagen matrix in the nascent DE is extensively modified by production and organization of human ECM components by the NIKS keratinocytes and NHDF.

A study was performed to characterize secretion of ECM molecules throughout the organotypic phase of StrataGraft® tissue manufacturing process. StrataGraft® skin tissue was demonstrated to synthesize fibrillar collagens types I and III, representing the predominant structural components of dermal ECM. The increase of antigen-specific staining by IIF throughout StrataGraft® skin tissue maturation demonstrates neosynthesis of human collagens. Synthesis of human type I collagen was further confirmed by detection of type I collagen C-propeptide (CICP), a specific metabolite associated with biosynthetic processing of type I collagen. Additional data demonstrate that human collagens are deposited and organized by the cellular components of StrataGraft®, including accumulation of type VI collagen and decorin, both of which function in vivo to guide assembly of collagen structures. StrataGraft® was demonstrated to organize a basement membrane zone and dermal-epidermal junction, as indicated by appropriate spatial distribution of collagen IV and laminin 332 (laminin 5). Absence of these ECM molecules in freshly poured DE indicates that the cellular components of StrataGraft® transform the nascent DE during the StrataGraft® maturation process to include human ECM components with central roles in the structural and signaling functions of skin tissue.

### Example 1—Indirect Immunofluorescence (IIF)

IIF was used to detect the ECM proteins in cryosections of the nascent, cellularized fresh DE as well as in developing tissues as various manufacturing stages. Due to paucity of NHDFs within the nascent DE, nuclear staining could not be visualized by fluorescence imaging using a DAPI nuclear counterstain. In lieu of this, a primary antibody against murine type I collagen, the primary ECM component in freshly poured DE was used. Counterstaining in this manner allowed for verification of the integrity of the DE.

### Type I Collagen

Type I collagen is a fibril-forming collagen that constitutes the major structural component of dermal ECM. Antibodies used for detection of type I collagen were directed against human and bovine type I collagen, and were highly cross-adsorbed against other human collagen types, serum proteins, and noncollagenous ECM proteins in order to increase antibody specificity. This antibody preparation is noted by the manufacturer to cross-react with murine type I collagen. Therefore, use of this reagent was not expected to distinguish between murine and human collagen within the developing tissue. Diffuse staining for type I collagen was present in freshly poured nascent DEs, confirming cross-reactivity with the murine type I collagen starting material. By Process Day 18, in addition to diffuse staining of collagen type I throughout the nascent DE, concentrated punctate staining was also evident throughout the nascent DE, frequently adjacent to nuclei from the NHDF which were captured within the tissue cross section.

By Process Day 33 (representing the notional drug substance immediately before cryopreservation), collagen type I expression was seen throughout the DE in a fibrillar distribution. In immediate post-thaw StrataGraft® drug product, localization of type I collagen appeared to be uniform throughout the dermal equivalent, likely a function of epitope unmasking resulting from reversible dissociation of collagen ECM by the glycerol component of the cryopreservation solution (Yeh, 2013). The change in staining patterns during tissue maturation is consistent with a shift from staining of the nascent, cellularized, murine collagen, gelled DE to show punctate staining of newly synthesized human type I collagen by the NHDF, followed by organization of the type I collagen into fibrils. Although the type I antibody used cannot distinguish between human and murine collagen, co-localization of type I collagen staining with type III collagen and decorin (see below) strongly supports human type I collagen biosynthesis during StrataGraft® skin tissue manufacture.

### Type III Collagen

Type III collagen is one of the major fibrillar collagens of elastic tissues, and is found to co-localize with tissues that are rich in type I collagen, due to assembly of heterotypic collagen fibrils containing both collagen types (Nystrom, 2019). The antibody preparation used for detection of this protein was raised against human type III collagen, and is reported to react with conformational determinants on type III collagen. This antibody preparation was cross adsorbed against human collagen types I, II, IV, V, and VI in order to eliminate cross-reactivity against these proteins. The antibody preparation is reported to cross-react with murine type III collagen. However, no type III collagen signal was detected in freshly poured DE, suggesting that murine type III collagen is not a significant component of the collagen starting material used in the formulation of nascent, cellularized DE. By Process Day 18, punctate staining was present throughout the DE that is often associated with the NHDF nuclei, as was noted with type I collagen. This localization is consistent with synthesis and secretion of type III collagen by the NHDF. Staining intensity increased throughout tissue maturation. This confirms the synthesis and deposition of human type III collagen during StrataGraft® skin tissue maturation.

### Type VI Collagen

Type VI collagen is a nonfibrillar collagen that comprises a filamentous meshwork within the ECM of native human skin, and has functional interactions with a variety of structural ECM components, including fibrillar collagen types I and III, type IV collagen, and decorin. It acts as a repository for growth factors and enzymes associated with wound healing and a regulator of dermal matrix assembly and composition (Nystrom, 2019). Type VI collagen has also been shown to precede the deposition of the major interstitial collagen types I and III during formation of ECM (Nystrom, 2019). The antibody preparation used for detection of human type VI collagen reacts with conformational determinants on native type VI collagen, does not recognize murine type VI collagen, and does not cross-react with human collagen types I, II, III, IV, or V. Type VI collagen was undetectable in freshly poured nascent DE. By Process Day 18, punctate staining was located in the dermal compartment, mirroring expression of types I and III collagen. By Process Day 33, type VI collagen was detected throughout the DE, confirming human collagen VI biosynthesis by the cellular components of StrataGraft® tissue.

### Decorin

Decorin is a dermatan sulfate proteoglycan associated with type I collagen fibril assembly, and has been used to visualize newly synthesized type I collagen both in vitro and in vivo (Oostendorp, 2016). The antibody preparation was raised against a recombinant fragment of the human decorin core protein and is reported to detect human decorin with potential murine cross reactivity.

Decorin was undetectable in freshly poured nascent DE but accumulated in the dermal compartment as StrataGraft® skin tissue matured, confirming synthesis of human decorin by the cellular components of StrataGraft® tissue during manufacture.

### Type IV Collagen

Type IV collagen (FIG. 6) is a major structural component of the dermal-epidermal junction within the basement membrane zone (Nystrom, 2019). The antibody preparation employed in this study was raised against purified human type IV collagen, and cross-reactivity with murine type IV collagen was not evaluated by the manufacturer. Type IV collagen was not detected in the nascent DE or during the early organotypic phases of StrataGraft® maturation. Expression of type IV collagen was detectable at the junction between the dermal and epidermal compartments at Process Day 33 of StrataGraft® skin tissue manufacture and in the cryopreserved final product, confirming that type IV collagen is synthesized and organized appropriately by cellular components of StrataGraft® tissue.

### Laminin 332 (Laminin 5)

Laminin 5 is found in the basement membrane zone of the skin, and is associated with anchoring filaments that contribute to adhesion of the epidermis to the underlying dermis (Nystrom, 2019). The antibody preparation utilized in this study was raised against a recombinant fragment of the human γ2 chain of laminin. This antibody preparation does not react with other laminin isoforms, however cross-reactivity against murine laminin 5 was not evaluated by the manufacturer. Laminin 5 was undetectable in freshly poured, nascent DE. However, by Process Day 18, punctate staining was apparent at the dermal-epidermal junction. By Process Day 26, contiguous staining was detected at the dermal-epidermal junction. This staining remained throughout tissue maturation, and by Process Day 33 expanded to include diffuse staining throughout the dermal compartment. These observations demonstrate that human laminin 5 is synthesized and deposited by the cellular components of StrataGraft® tissue. In Process Day 33 and post-thaw specimens, this antibody preparation was noted to bind nonspecifically to this polycarbonate membrane, resulting in a signal associated with the membrane itself.

### Example 2—Quantification of Human Collagen In-Process and in the Final Drug Product by ELISA Analysis

Synthesis of human type I collagen was independently confirmed using an ELISA specific to human cross-linked C-telopeptides of Type 1 collagen (CICP). Input murine type I collagen used in DE formulation consists of mature collagen that contains no CICP. Newly synthesized type I collagen is produced within cells as procollagen, which is not competent to polymerize into fibrillar collagen structures. Upon secretion, procollagen is converted to tropocollagen by specific extracellular proteases to release both N- and C-terminal propeptides, allowing for polymerization of fibrillar collagen structures. The released CICP is a marker of type I collagen biosynthesis (Parfitt, 1987), and can be quantitated in order to obtain a minimum rate of collagen biosynthesis.

Human CICP was quantitated in conditioned media collected from StrataGraft® tissues immediately prior to NIKS seed, and at each media change up to the cryopreservation step. Samples were tested and compared against a CICP reference standard curve using a human-specific CICP ELISA. Results were expressed as [m/(v*t)], or mass (m) per unit volume (v) of media per day (t). Minimum rate of synthesis of human collagen I was extrapolated from measured values based on a 1:1 molar ratio of type I CICP trimer and tropocollagen I trimer.

Results of ELISA and quantitative estimates of human type I collagen are shown in Table 1. Total secreted CICP was determined by multiplying the average CICP content by the total media volume at each process step. CICP secretion rate was obtained by dividing the total secreted CICP by the tissue area and culture interval between media replacement. Minimum estimates of type I collagen synthesis were obtained by calculating the predicted molecular weight of the CICP and mature tropocollagen trimers and, based on a 1:1 stoichiometry between CICP trimer and mature tropocollagen trimer, multiplying the CICP secretion rate by the mass ratio of tropocollagen to CICP. Cumulative human type I collagen was obtained by multiplying the minimum collagen synthesis rate by total tissue area and culture interval for each process step, and summing the obtained values.

TABLE 1
Synthesis of Human Type I Collagen by StrataGraft ® tissue

| | | | | | Minimum | |
|---|---|---|---|---|---|---|
| | | | | | Type I | |
| Process | | | | CICP | collagen | Cumulative |
| Step at Media | Average | Media | Secreted | secretion | synthesis | Type I |
| Collection | CICP (ng/mL) | Volume (mL) | CICP (ng) | (ng/cm²/day) | (ng/cm²/day) | Collagen (mg) |
| NIKS Seed | 162 | 268 | 43422 | 87 | 320 | 0.160 |
| Process Day 15 | 240 | 245 | 58838 | 294 | 1086 | 0.377 |
| Process Day 18 | 1088 | 200 | 217696 | 726 | 2678 | 1.18 |
| Process Day 22 | 1694 | 200 | 338847 | 847 | 3126 | 2.43 |
| Process Day 26 | 1729 | 200 | 345794 | 864 | 3190 | 3.71 |
| Process Day 29 | 1258^{a} | 200 | 251645 | 839 | 3095 | 4.64 |
| Cryopreservation | 1541^{a} | 200 | 308117 | 770 | 2842 | 5.77 |

^{a}N = 1 (One set of samples was invalidated because values fell outside the standard curve)

Type I collagen synthesis was detectable at low levels prior to seed of NIKS keratinocytes on the dermal equivalent. During early organotypic phases (NIKS seed and Process Day 15) NIKS keratinocytes proliferate and spread over the surface of the nascent DE. Once an air-liquid interface is initiated at Process Day 15, paracrine signaling between NIKS and NHDFs is anticipated to result in significant increase in CICP production, and peak levels of collagen synthesis can be inferred from elevated levels seen in conditioned media collected prior to Process Day 26. The data support that a minimum of approximately 5.8 mg of human type I collagen is synthesized and incorporated as mature protein into the mature DE of the drug product.

### Example 3—Quantification of Total Collagen in StrataGraft® Skin Tissue

The CICP data discussed above demonstrated in vitro biosynthesis of human type I collagen during the StrataGraft® manufacturing process, and provided an estimated amount of human type I collagen produced in StrataGraft® skin tissue to be at least 5.8 mg per tissue. Additional studies were performed to quantify the total collagen content in StrataGraft® tissue by utilizing the unique amino acid composition of mammalian collagens. Both murine and human type I collagen are comprised of 12-14% hydroxyproline by mass, in order to create an amino acid structure that is compatible with collagen assembly (Stoilov, 2018).

The study utilized a colorimetric assay kit for measurement of total hydroxyproline, which was then used to quantify total collagen in the StrataGraft® skin tissue. Residual murine collagen was then estimated by subtracting the estimated human collagen component obtained from the CICP ELISA from total measured collagen. From the estimated biosynthesis of human collagen, compared to total collagen in the final product, it was possible to approximate the relative contribution of murine type I collagen and human type I collagen in StrataGraft® tissue.

Samples were subjected to alkaline hydrolysis, and subsequently neutralized. Experimental samples and collagen reference samples were transferred into the wells of a 96-well microplate. Hydroxyproline content was measured using a Hydroxyproline Assay Kit (perchlorate-free) (BioVision, Inc., Milpitas, Calif.). Hydroxyproline standard solution (1 mg/mL) was diluted to 0.1 mg/mL and applied in triplicate to 96-well microwell plates in order to generate a standard curve ranging from 0 to 1 μg hydroxyproline per sample well. Hydrolysates and standards were evaporated to dryness, oxidized with Chloramine T, and reacted with a developer solution containing DMAB (Ehrlich's reagent). Absorbance measurements were taken at 560 nm to determine hydroxyproline content, using a SpectraMax Plus plate reader (Molecular Devices), using SoftMax Pro software package v.7.0.2.

The feasibility of the hydroxyproline assay was initially verified by assessing the ability of the assay to quantify total collagen in samples consisting of both murine collagen and human collagen. Samples of murine type I collagen were diluted to 1.0 mg/mL and mixed with increasing amounts of purified human fibroblast-derived type I collagen that had been diluted to 1.0 mg/mL (Table 2). Samples were used as reference standards in order to verify that the method provides comparable readouts for both human and murine type I collagen.

TABLE 2
Titration of Murine Collagen with Human Type I Collagen

| | Volume 1.0 mg/mL | Volume 1.0 mg/mL |
|---|---|---|
| Sample ID | murine type I | Human type I collagen |
| RCI-100 | 1.0 | 0 |
| RCI-90 | 0.9 | 0.1 |
| RCI-80 | 0.8 | 0.2 |
| RCI-50 | 0.5 | 0.5 |
| RCI-20 | 0.2 | 0.8 |
| RCI-0 | 0 | 1.0 |

A summary of the measured titration samples is provided in Table 3. There were no apparent differences in total collagen content across reference samples containing various levels of murine and human type I collagen, as indicated by the flat slope of the trend line derived from a scatterplot of total collagen vs. % rat collagen in the reference samples (−0.0004). This data confirms that hydroxyproline content in Type I collagen is consistent between murine and human collagen.

TABLE 3
Quantification of Hydroxyproline in Collagen Reference Samples

| | Input | [HypL] (μg | Measured collagen |
|---|---|---|---|
| | collagen | hydroxyproline | in reference |
| Sample | (mg) | per mL sample) | sample (mg) |
| RCI-100 | 1.0 | 0.1362 | 1.01 |
| RCI-90 | 1.0 | 0.1563 | 1.16 |
| RCI-80 | 1.0 | 0.1296 | 0.96 |
| RCI-50 | 1.0 | 0.1566 | 1.16 |
| RCI-20 | 1.0 | 0.1446 | 1.07 |
| RCI-0 | 1.0 | 0.1446 | 1.07 |

Two cryopreserved tissues from each of three lots of StrataGraft® tissue were evaluated for total collagen content. Tissues were manufactured at the Stratatech Corp., 535 Science Drive manufacturing facility at the commercial 80-tissue lot scale (StrataGraft® lots FP0001-0000088596 and FP0001-0000088597) or in the company's Process Development laboratory at 510 Charmany Drive at 20-tissue lot scale (lot SG-C100-102419-67) using murine collagen from a single manufacturing lot (Corning). Tissues were thawed at ambient temperature and transferred into a hold dish containing 15 mL of Stratatech Hold Solution that had been warmed to 35 to 39° C. Tissues were held at ambient room temperature for at least 15 minutes to allow for removal of cryoprotectant solution. Samples of the tissues were harvested, homogenized and subjected to analysis of hydroxyproline content. A summary of tissues and test samples under evaluation is provided in Table 4.

TABLE 4
Hydroxyproline Assay-Samples under Test

| | Age of Collagen | Age of Tissues | | |
|---|---|---|---|---|
| Tissue Lot | at Point of Use | at Analysis | Tissue # | Sample |
| FP0001-0000088596 | 6 months | 9 months | 7 | A |
| | | | | B |
| | | | 8 | A |
| | | | | B |
| FP0001-0000088597 | 6 months | 9 months | 7 | A |
| | | | | B |
| | | | 6 | A |
| | | | | B |
| SG-C100-102419-67 | 12 months | 3 months | 15 | A |
| | | | | B |
| | | | 14 | A |
| | | | | B |

Table 5 summarizes the results from StrataGraft® test samples. Based on reported values of 13.5% hydroxyproline by mass, collagen content in StrataGraft® tissue was estimated at 29.6 to 38.6 mg per tissue (mean value 32.9 mg; n=6 tissues). Based on the estimate of human collagen type I of 5.8 mg per tissue by CICP ELISA present in StrataGraft® skin tissue final product, 15-20% of collagen by mass is derived from NHDF and the remaining 80.4-85.0% of collagen by mass is of murine origin. Based on an input mass of 51.5 mg murine collagen, it is implied that approximately 37-54% of the input murine collagen may be eliminated during the StrataGraft® manufacturing process.

TABLE 5
Collagen Content in StrataGraft ® Skin Tissues

| | | Total Collagen | |
|---|---|---|---|
| | Hydroxyproline | Content | % human |
| Tissue | Content (μg per | (mg per tissue) | collagen |
| FP0001-0000088596 | 4070 | 30.2 | 19.3 |
| FP0001-0000088596 | 3998 | 29.6 | 19.6 |
| FP0001-0000088597 | 5207 | 38.6 | 15.0 |
| FP0001-0000088597 | 4376 | 32.4 | 17.9 |
| SG-C100-102419-67 | 4325 | 32.0 | 18.1 |
| SG-C100-102419-67 | 4643 | 34.4 | 16.9 |

### Example 4—Contribution of Collagen Component to StrataGraft® Mechanical Properties

Importantly, the exogenous murine collagen gel provides an initial physiological substrate for the input NHDF and NIKS cells, but does not contribute substantially to the final mechanical properties of the tissues. Mechanical properties of tissues were evaluated by uniaxial tensile tests. Standard dog bone-shaped tensile specimens (4 mm gauge width, 25 mm gage length) were cut from the tissues using a stainless steel die and a manual toggle press. Thickness measurements were taken using a Mitutoyo digital thickness gauge. Specimens were pulled to failure in uniaxial tension at a constant strain rate of 100% per minute on an Insight 1 Bionix tensiometer (MTS Systems, Eden Prairie, Minn.) with continuous hydration with DPBS. Peak load at failure was determined from load and displacement data acquired using Testworks 4 software.

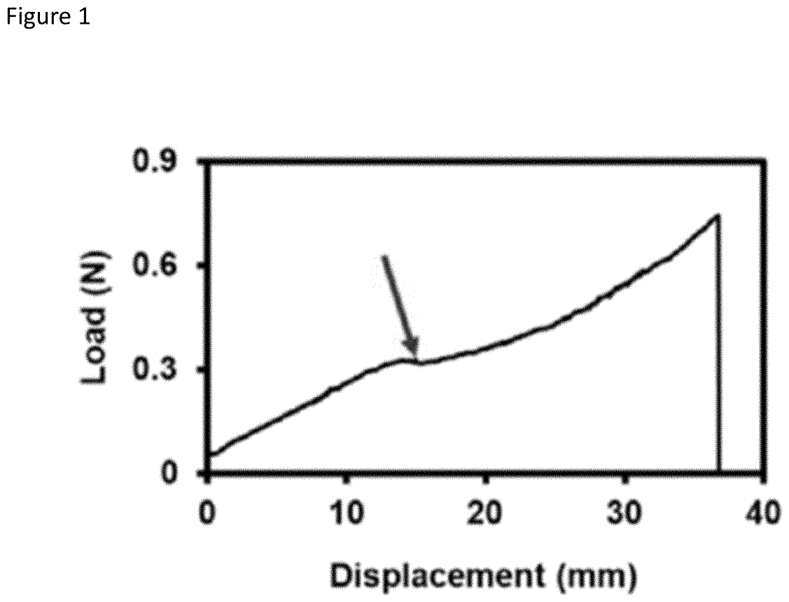

Visual observation of tensile specimens during testing identified that StrataGraft® tissues fail in two distinct phases in tension, with the DE breaking first and then the sample failing when the epidermal layer breaks at a higher load and displacement. FIG. 1 shows a representative load displacement curve, with the point of the DE failure marked by the arrow.

The profile of the load displacement curve demonstrates that there is minimal contribution of the DE layer to the overall tissue tensile properties. If the DE contributed significant mechanical strength to the tissue, there would be a pronounced drop in load at the point where it breaks, rather than the slight plateau in the load-displacement curve. The drop in load associated with DE failure from 12 StrataGraft® samples evaluated as part of a comparability study resulted in an average drop of just 6 mN, or approximately 1% of the overall failure load of the tissue. There would also be a significant difference in the slopes of the load-displacement curve before and after the break; instead the curve has similar slopes both before and after the DE failure, suggesting that the resistance to sample elongation is coming almost exclusively from the epidermal layer.

### ENUMERATED EMBODIMENTS

Embodiment 1. A viable, bioengineered skin construct comprising
a fully stratified epithelial layer having a top surface and a bottom surface, wherein the fully stratified epithelial layer comprises human keratinocytes;
a dermal equivalent layer having a top surface and a bottom surface, wherein the dermal equivalent layer comprises human dermal fibroblasts within a matrix, the matrix comprising human collagen and optionally murine type I collagen;

wherein the bottom surface of the epithelial layer is adhered to the top surface of the dermal equivalent layer over at least 98% of one of the layers; and

wherein a dog bone-shaped sample of the skin construct fails at a load of about 0.5 N to about 1.0 N (“the failure load”) and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm;

wherein the dog bone-shaped sample has a gauge width of 4 mm and a gauge length of 25 mm, as measured by a thickness gauge.

Embodiment 2. The skin construct of embodiment 1, wherein the load drops less than 10% of the failure load when the dermal equivalent layer fails.

Embodiment 3. The skin construct of embodiment 2, wherein the load drops less than 5% of the failure load when the dermal equivalent layer fails.

Embodiment 4. The skin construct of embodiment 2, wherein the load drops less than 1% of the failure load when the dermal equivalent layer fails.

Embodiment 5. The skin construct of any one of embodiments 1 to 4, wherein the dermal equivalent layer fails at a displacement of about 14-16 mm and a load of about 0.1 N to about 0.5 N.

Embodiment 6. The skin construct of any one of embodiments 1 to 5, wherein the skin construct has a thickness of about 100 μm to about 250 μm, or about 120 μm to about 200 μm, as measured by histology.

Embodiment 7. The skin construct of embodiment 6, wherein the fully stratified epithelial layer has a thickness of about 75 μm to about 120 μm and/or the dermal equivalent layer has a thickness of about 20 μm to about 80 μm, as measured by histology.

Embodiment 8. The skin construct of any one of embodiments 1 to 7, wherein the keratinocytes of the epithelial layer are from a single human donor and/or the dermal fibroblasts of the dermal equivalent layer are from a single human donor, optionally different from the human donor of the keratinocytes.

Embodiment 9. The skin construct of embodiment 8, wherein the keratinocytes are NIKS cells or the dermal fibroblasts are normal human dermal fibroblasts.

Embodiment 10. The skin construct of embodiment 8, wherein the keratinocytes are NIKS cells and the dermal fibroblasts are normal human dermal fibroblasts.

Embodiment 11. The skin construct of any one of the preceding embodiments, wherein the skin construct has a surface area of about 40 cm² to about 100 cm².

Embodiment 12. The skin construct of any one of the preceding embodiments, wherein the skin construct comprises human type I collagen.

Embodiment 13. The skin construct of embodiments 12, wherein the skin construct has at least 5 mg of human type I collagen.

Embodiment 14. The skin construct of embodiment 12, wherein the skin construct has at least 5.5 mg of human type I collagen or at least at least 5.8 mg of human type I collagen.

Embodiment 15. The skin construct of any one of embodiments 12 to 14, wherein about 98% or more of the human type I collagen is produced by cells of the skin construct.

Embodiment 16. The skin construct of embodiment 15, wherein about 100% of the human type I collagen is produced by cells of the skin construct.

Embodiment 17. The skin construct of any one of embodiments 12 to 16, wherein the skin construct comprises human type I collagen and murine type I collagen, wherein the murine type I collagen is not more than 90% by weight of total collagen in the skin construct.

Embodiment 18. The skin construct of any one of embodiments 12 to 17, wherein the skin construct comprises human type I collagen and murine type I collagen, wherein the murine type 1 collagen is about 60% to about 90% by weight of the total type collagen in the skin construct.

Embodiment 19. The skin construct of any one of the preceding embodiments, wherein the skin construct has a total collagen content of about 0.25 mg to about 0.45 mg per cm² of surface area, or about 0.29 mg to about 0.39 mg per cm² of surface area.

Embodiment 20. A viable, bioengineered skin construct comprising
a fully stratified epithelial layer having a top surface and a bottom surface, wherein the fully stratified epithelial layer comprises human keratinocytes;
a dermal equivalent layer having a top surface and a bottom surface, wherein the bottom surface of the epithelial layer is adhered to the top surface of the dermal equivalent layer over at least 98% of one of the layers,
   wherein the dermal equivalent layer comprises human dermal fibroblasts within a matrix, the matrix comprising human type I collagen, human type III collagen, human type IV collagen, and human type VI collagen and optionally murine type I collagen; and
a total collagen content of about 0.25 mg per cm² of surface area to about 0.45 mg per cm² of surface area and at least 0.05 mg per cm² of surface area of human type I collagen;

wherein a dog bone-shaped sample of the skin construct fails at a load of about 0.5 N to about 1.0 N and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm,

wherein the dog bone-shaped sample has a gauge width of 4 mm and a gauge length of 25 mm, as measured by a thickness gauge.

Embodiment 21. The skin construct of embodiment 20, wherein the keratinocytes of the epithelial layer are from a single human donor and/or the dermal fibroblasts of the dermal equivalent layer are from a single human donor, optionally different from the human donor of the keratinocytes.

Embodiment 22. The skin construct of embodiment 21, wherein the keratinocytes are NIKS cells or the dermal fibroblasts are normal human dermal fibroblasts.

Embodiment 23. The skin construct of embodiment 21, wherein the keratinocytes are NIKS cells and the dermal fibroblasts are normal human dermal fibroblasts.

Embodiment 24. A viable, bioengineered skin construct comprising
a fully stratified epithelial layer having a top surface and a bottom surface, wherein the fully stratified epithelial layer comprises NIKS cells;
a dermal equivalent layer having a top surface and a bottom surface, wherein the bottom surface of the epithelial layer is adhered to the top surface of the dermal equivalent layer over at least 98% of one of the layers,
wherein the dermal equivalent layer comprises normal human dermal fibroblasts within a matrix, the matrix comprising human type I collagen, human type III collagen, human type IV collagen, and human type VI collagen and optionally murine type I collagen; and
a total collagen content of about 0.25 mg per cm² of surface area to about 0.45 mg per cm² of surface area and at least 0.05 mg per cm² of surface area of human type I collagen;

wherein a dog bone-shaped sample of the skin construct fails at a load of about 0.5 N to about 1.0 N and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm

wherein the dog bone-shaped sample has a gauge width of 4 mm and a gauge length of 25 mm, as measured by a thickness gauge.

Embodiment 25. The skin construct of any one of embodiments 20-24, wherein the load drops less than 10% of the failure load when the dermal equivalent layer fails.

Embodiment 26. The skin construct of embodiment 25, wherein the load drops less than 5% of the failure load when the dermal equivalent layer fails.

Embodiment 27. The skin construct of embodiment 25, the load drops less than 1% of the failure load when the dermal equivalent layer fails.

Embodiment 28. The skin construct of any one of embodiments 20 to 27, wherein the dermal equivalent layer fails at a displacement of about 14-16 mm and a load of about 0.1 N to about 0.5 N.

Embodiment 29. The skin construct of any one of embodiments 20 to 28, wherein the skin construct has a thickness of about 100 μm to about 250 μm, or about 120 μm to about 200 μm, as measured by histology.

Embodiment 30. The bioengineered skin construct of embodiment 29, wherein the fully stratified epithelial layer has a thickness of about 75 μm to about 120 μm and/or the dermal equivalent layer has a thickness of about 20 μm to about 80 μm, as measured by histology.

Embodiment 31. The skin construct of any one of embodiments 20 to 30, wherein the skin construct has at least 0.055 mg of human type I collagen per cm² of surface area or at least at least 0.058 mg of human type I collagen per cm² of surface area.

Embodiment 32. The skin construct of any one of embodiments 20 to 31, wherein about 98% or more of the human type I collagen is produced by cells of the skin construct.

Embodiment 33. The skin construct of embodiment 32, wherein about 100% of the human type I collagen is produced by cells of the skin construct.

Embodiment 34. The skin construct of any one of embodiments 20 to 33, wherein the total collagen content is about 0.29 mg per cm² of surface area to about 0.39 mg per cm² of surface area.

Embodiment 35. The skin construct of any one of embodiments 20 to 34, wherein the skin construct comprises human type I collagen and murine type I collagen, wherein the murine type I collagen is not more than 90% by weight of total collagen in the skin construct.

Embodiment 36. The skin construct of any one of embodiments 20 to 34, wherein the skin construct comprises human type I collagen and murine type I collagen, wherein the murine type 1 collagen is about 60% to about 90% by weight of the total type collagen in the skin construct.

Embodiment 37. The bioengineered skin construct of any one of embodiments 20 to 36, wherein the skin construct has a surface area of about 40 cm² to about 100 cm².

Embodiment 38. A viable, bioengineered skin construct comprising
a fully stratified epithelial layer having a top surface and a bottom surface, wherein the fully stratified epithelial layer comprises human keratinocytes;
a dermal equivalent layer having a top surface and a bottom surface, wherein the bottom surface of the epithelial layer is adhered to the top surface of the dermal equivalent layer over at least 98% of one of the layers,
   wherein the dermal equivalent layer comprises human dermal fibroblasts within a matrix, the matrix comprising human type I collagen, human type III collagen, human type IV collagen, and human type VI collagen and optionally murine type I collagen; and

a total collagen content of about 0.25 mg per cm² of surface area to about 0.45 mg per cm² of surface area and at least 0.05 mg per cm² of surface area of human type I collagen.

Embodiment 39. The skin construct of embodiment 38, wherein the keratinocytes of the epithelial layer are from a single human donor and/or the dermal fibroblasts of the dermal equivalent layer are from a single human donor, optionally different from the human donor of the keratinocytes.

Embodiment 40. The skin construct of embodiment 39, wherein the keratinocytes are NIKS cells or the dermal fibroblasts are normal human dermal fibroblasts.

Embodiment 41. The skin construct of embodiment 39, wherein the keratinocytes are NIKS cells and the dermal fibroblasts are normal human dermal fibroblasts.

Embodiment 42. A viable, bioengineered skin construct comprising
a fully stratified epithelial layer having a top surface and a bottom surface, wherein the fully stratified epithelial layer comprises NIKS cells;
a dermal equivalent layer having a top surface and a bottom surface, wherein the bottom surface of the epithelial layer is adhered to the top surface of the dermal equivalent layer over at least 98% of one of the layers,
   wherein the dermal equivalent layer comprises normal human dermal fibroblasts within a matrix, the matrix comprising human type I collagen, human type III collagen, human type IV collagen, and human type VI collagen and optionally murine type I collagen; and

a total collagen content of about 0.25 mg per cm² of surface area to about 0.45 mg per cm² of surface area and at least 0.05 mg per cm² of surface area of human type I collagen.

Embodiment 43. The skin construct of any one of embodiments 38 to 42, wherein the skin construct has at least 0.055 mg per cm² of surface area of human type I collagen or at least 0.058 mg per cm² of surface area of human type I collagen.

Embodiment 44. The skin construct of any one of embodiments 38 to 43, wherein about 98% or more of the human type I collagen is produced by cells of the skin construct.

Embodiment 45. The skin construct of embodiment 44, wherein about 100% of the human type I collagen is produced by cells of the skin construct.

Embodiment 46. The skin construct of any one of embodiments 38 to 46, wherein the skin construct comprises human type I collagen and murine type I collagen, wherein the murine type I collagen is not more than 90% by weight of total collagen in the skin construct.

Embodiment 47. The skin construct of any one of embodiments 38 to 46, wherein the skin construct comprises human type I collagen and murine type I collagen, wherein the murine type 1 collagen is about 60% to about 90% by weight of the total type collagen in the skin construct.

Embodiment 48. The skin construct of any one of embodiments 38 to 47, wherein the skin construct has a thickness of about 100 μm to about 250 μm, or about 120 μm to about 200 μm, as measured by histology.

Embodiment 49. The skin construct of embodiment 48, wherein the fully stratified epithelial layer has a thickness of about 75 μm to about 120 μm and/or the dermal equivalent layer has a thickness of about 20 μm to about 80 μm, as measured by histology.

Embodiment 50. The bioengineered skin construct of any one of embodiments 38 to 49, wherein the skin construct has a surface area of about 40 cm² to about 100 cm².

Embodiment 51. The skin construct of any one of embodiments 38 to 50, wherein a dog bone-shaped sample of the skin construct fails at a load of about 0.5 N to about 1.0 N and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm

wherein the dog bone-shaped sample has a gauge width of 4 mm and a gauge length of 25 mm, as measured by a thickness gauge.

Embodiment 52. The skin construct of embodiment 51, the load drops less than 10% of the failure load when the dermal equivalent layer fails.

Embodiment 53. The skin construct of embodiment 52, the load drops less than 5% of the failure load when the dermal equivalent layer fails.

Embodiment 54. The skin construct of embodiment 53, the load drops less than 1% of the failure load when the dermal equivalent layer fails.

Embodiment 55. The skin construct of any one of embodiments 51 to 54, wherein the dermal equivalent layer fails at a displacement of about 14-16 mm and a load of about 0.1 N to about 0.5 N.

Embodiment 56. The skin construct of any one of the preceding embodiments, wherein the skin construct is cryopreserved.

Embodiment 57. The skin construct of embodiment 56, wherein the skin construct, after thawing, secretes a plurality of proteins selected from bFGF, GM-CSF, HGF, IL-1α, IL-6, IL-8, IL-10, MMP-1, MMP-3, MMP-9, PIGF, SDF-1α, TGF-β1, and VEGF-A.

What is claimed is:

1. A viable, bioengineered skin construct comprising
a fully stratified epithelial layer having a top surface and a bottom surface, wherein the fully stratified epithelial layer comprises human keratinocytes;
a dermal equivalent layer having a top surface and a bottom surface, wherein the dermal equivalent layer comprises human dermal fibroblasts within a matrix, the matrix comprising human collagen and optionally murine type I collagen;
wherein the bottom surface of the epithelial layer is adhered to the top surface of the dermal equivalent layer over at least 98% of one of the layers; and
wherein a dog bone-shaped sample of the skin construct fails at a load of about 0.5 N to about 1.0 N (“the failure load”) and a displacement of about 30 mm to about 45 mm when pulled to failure in uniaxial tension at a constant strain rate of 100% per minute with continuous hydration using Dulbecco's phosphate-buffered saline, and wherein the fully stratified epithelial layer fails at the same point in the load-displacement curve as the skin construct and the dermal equivalent layer fails at a displacement of about 12-18 mm or about 14-16 mm;
wherein the dog bone-shaped sample has a gauge width of 4 mm and a gauge length of 25 mm, as measured by a thickness gauge.

2. The skin construct of claim 1, wherein the load drops less than 10% of the failure load when the dermal equivalent layer fails.

3. The skin construct of claim 2, wherein the load drops less than 5% of the failure load when the dermal equivalent layer fails.

4. The skin construct of claim 2, wherein the load drops less than 1% of the failure load when the dermal equivalent layer fails.

5. The skin construct of claim 1, wherein the dermal equivalent layer fails at a displacement of about 14-16 mm and a load of about 0.1 N to about 0.5 N.

6. The skin construct of claim 1, wherein the skin construct has a thickness of about 100 μm to about 250 μm, or about 120 μm to about 200 μm, as measured by histology.

7. The skin construct of claim 6, wherein the fully stratified epithelial layer has a thickness of about 75 μm to about 120 μm and/or the dermal equivalent layer has a thickness of about 20 μm to about 80 μm, as measured by histology.

8. The skin construct of claim 1, wherein the keratinocytes of the epithelial layer are from a single human donor and/or the dermal fibroblasts of the dermal equivalent layer are from a single human donor, optionally different from the human donor of the keratinocytes.

9. The skin construct of claim 8, wherein the keratinocytes are NIKS cells or the dermal fibroblasts are normal human dermal fibroblasts.

10. The skin construct of claim 8, wherein the keratinocytes are NIKS cells and the dermal fibroblasts are normal human dermal fibroblasts.

11. The skin construct of claim 1, wherein the skin construct has a surface area of about 40 cm² to about 100 cm².

12. The skin construct of claim 11, wherein the skin construct comprises human type I collagen.

13. The skin construct of claim 12, wherein the skin construct has at least 5 mg of human type I collagen.

14. The skin construct of claim 12, wherein the skin construct has at least 5.5 mg of human type I collagen or at least at least 5.8 mg of human type I collagen.

15. The skin construct of claim 1, wherein about 98% or more of the human type I collagen is produced by cells of the skin construct.

16. The skin construct of claim 15, wherein about 100% of the human type I collagen is produced by cells of the skin construct.

17. The skin construct of claim 12, wherein the skin construct comprises human type I collagen and murine type I collagen, wherein the murine type I collagen is not more than 90% by weight of total collagen in the skin construct.

18. The skin construct of claim 12, wherein the skin construct comprises human type I collagen and murine type I collagen, wherein the murine type 1 collagen is about 60% to about 90% by weight of the total type collagen in the skin construct.

19. The skin construct of claim 1, wherein the skin construct has a total collagen content of about 0.25 mg to about 0.45 mg per cm² of surface area, or about 0.29 mg to about 0.39 mg per cm² of surface area.

20. A viable, bioengineered skin construct comprising
a fully stratified epithelial layer having a top surface and a bottom surface, wherein the fully stratified epithelial layer comprises human keratinocytes;
a dermal equivalent layer having a top surface and a bottom surface, wherein the bottom surface of the epithelial layer is adhered to the top surface of the dermal equivalent layer over at least 98% of one of the layers,
wherein the dermal equivalent layer comprises human dermal fibroblasts within a matrix, the matrix comprising human type I collagen, human type III collagen, human type IV collagen, and human type VI collagen and optionally murine type I collagen; and
a total collagen content of about 0.25 mg per cm² of surface area to about 0.45 mg per cm² of surface area and at least 0.05 mg per cm² of surface area of human type I collagen.
